(19) United States
(12) Patent Application Publication
(10) Pub. No.: US 2014/0037169 A1
(43) Pub. Date: Feb. 6, 2014

(54) METHOD FOR MOVEMENT-AVERAGED ATTENUATION CORRECTION AND MAGNETIC RESONANCE SYSTEM

(71) Applicant: SIEMENS AKTIENGESELLSCHAFT, Munich (DE)

(72) Inventor: Matthias FENCHEL, Erlangen (DE)

(73) Assignee: SIEMENS AKTIENGESELLSCHAFT, Munich (DE)

(21) Appl. No.: 13/943,941

(22) Filed: Jul. 17, 2013

(30) Foreign Application Priority Data
Aug. 1, 2012 (DE) 102012213551.0

(51) Int. Cl. A61B 5/00

(52) U.S. Cl. CPC A61B 5/0035
USPC 382/131; 600/411

(57) ABSTRACT

Various embodiments relate to a method for the movement-averaged attenuation correction of positron emission tomography data based on magnetic resonance tomography data. In at least one embodiment, the method includes capturing of multiple MRT data respectively in different phases of a cycle of an anatomical disposition of an investigation subject. The method furthermore includes respectively for each of the plurality of MRT data: determination of a value of an attenuation parameter via segmentation and averaging of the determined values of the attenuation parameter in order to obtain an averaged value of the attenuation parameter. The method furthermore includes the execution of attenuation correction of the PET data based on the averaged value of the attenuation parameter.

### PRIORITY STATEMENT

The present application hereby claims priority under 35 U.S.C. §119 to German patent application number DE 102012213551.0 filed Aug. 1, 2012, the entire contents of which are hereby incorporated herein by reference.

### FIELD

Various embodiments relate to a method for the movement-averaged attenuation correction of positron emission tomography data based on magnetic resonance tomography data and a magnetic resonance system. In particular, various embodiments relate to the preceding segmentation of the magnetic resonance tomography data for determining a value of an attenuation parameter and the subsequent averaging of the determined values of the attenuation parameter.

### BACKGROUND

Techniques are known which allow attenuation correction of (PET) data captured in positron emission tomography to be carried out on the basis of captured magnetic resonance tomography (MRT) data. As part of the attenuation correction, the attenuation of PET photons emitted due to an interaction between positrons and electrons is determined for the path of the PET photons through absorbing tissue to a PET detector of a PET imaging unit. The signal detected by the PET imaging unit is corrected in order to reduce or eliminate this determined attenuation in the PET data. The attenuation correction is typically based on an attenuation map (μ-map), which provides a linear attenuation parameter (μ) or absorption value of the PET photons in a spatially resolved manner.

The attenuation map (μ-map) may be determined on the basis of MRT data obtained by way of determined MRT measurement sequences and possibly postprocessing techniques. Such MRT measurement sequences may comprise Dixon-type MRT measurement sequences or chemical-shift imaging, such as are known to the person skilled in the art. In this case the corresponding MRT data is captured both for an investigation area and for an area surrounding the investigation area, these areas corresponding to the patient's anatomy along the path of the PET photons. The captured MRT data may subsequently be segmented. A possible postprocessing technique for segmentation is based on the MRT data captured by way of a Dixon-type MRT measurement sequence and segmented into fat, water, lungs and air. These segmentation classes may then be assigned different values of the attenuation parameter; this is because the attenuation of the PET photons varies characteristically for areas with fat, water, lungs and air.

In general the segmentation of MRT data for determining values of the attenuation parameter means: selection of values (in this case attenuation parameters) from a determined set (e.g. attenuation parameter values for fat, water, lungs, air) for the various pixels of the MRT data.

Alternative techniques for determining the values of the attenuation parameter are known, e.g. from A. V. Bronnikov “Reconstruction of Attenuation Map Using Discrete Consistency Conditions” in IEEE Trans. Med. Imag. 19 (2000) 451-462; or from J. Nuyts et al., “Completion of a Truncated Attenuation Image from the Attenuated PET Emission Data” in Nuclear Sci. Symp. Conf. Rec. (2010) 2123-2127, the entire contents of each of which are incorporated herein by reference.

One problem with attenuation correction is the extent to which a comparable coordination of the MRT data and/or of the values of the attenuation parameter on the one hand, and of the PET data on the other, may take place on the cyclical movement of the patient, e.g. during breathing or swallowing.

The PET data (e.g. depending on the activity of a radiopharmaceutical) is typically captured over a relatively long period, whilst MRT data may be captured within a relatively shorter period. Techniques are therefore known which allow the MRT data to be captured during a breath-holding phase of an investigation subject, e.g. before or after the PET data is captured.

It is however possible for the position of anatomical features to vary significantly between the MRT data captured in the breath-holding phase and the PET data, e.g. averaged over many respiration cycles. This may result in image artifacts. Examples of such artifacts include undercorrection of the liver or overcorrection of the lungs, if the position of the diaphragm varies between MRT data and PET data. A further class of artifacts, as they are known to the person skilled in the art, is known as “hot lung” artifacts, i.e. an overcorrection of the peripheral areas of the lungs.

Corresponding difficulties or artifacts may also occur analogously with the special attenuation correction techniques mentioned at the start, such as are known from the publications of A. V. Bronnikov and J. Nuyts et al.

Generally speaking, the accuracy of attenuation correction may be reduced and therefore image errors in the PET data increased, due to the respiration of the investigation subject or as a result of movement in general. Accuracy of the PET data is reduced. Subsequent applications (e.g. of a diagnostic type) may therefore be erroneous. It may be possible, for example, for certain values such as organ volumes, quantitative activity values, etc. only to be determined from the PET data with a relatively major error.

### SUMMARY

The inventors have discovered that a need exists for improved techniques for movement-averaged attenuation correction of PET data, based on MRT data, to be provided. In particular, they have recognized that there is a need to provide techniques which simultaneously allow PET and MRT data to be captured quickly and easily.

According to one aspect of at least one embodiment, the invention relates to a method for movement-averaged attenuation correction of positron emission tomography (PET) data of an investigation subject, based on magnetic resonance tomography (MRT) data. The method comprises the capture of multiple MRT data respectively for the investigation area and for an area surrounding the investigation area, in each case in different phases of a cycle of an anatomical disposition of the investigation subject. The method furthermore comprises, for each of the plurality of MRT data respectively, the determination of a value of an attenuation parameter from the respective MRT data by way of segmentation, this determination taking place in a spatially resolved manner for the investigation area and the surrounding area. The method furthermore comprises the averaging of the determined values of the attenuation parameter in order to obtain an averaged value of the attenuation parameter, the averaging taking place in a spatially resolved manner for the investigation area and the surrounding area. The method furthermore comprises the execution of attenuation correction of the PET data, based on the averaged value for the attenuation parameter.

According to a further aspect, at least one embodiment of the invention relates to an MRT system for movement-averaged attenuation correction of PET data of an investigation area of an investigation subject, based on MRT data, the MRT system comprising an MRT imaging unit and a processor unit. The MRT imaging unit is configured to capture multiple MRT data respectively for the investigation area and an area surrounding the investigation area and respectively in different phases of a cycle of the anatomical disposition of the investigation subject. The processor unit is configured to carry out the following steps: respectively for each of the plurality of MRT data: determination of a value of an attenuation parameter from the respective MRT data by means of segmentation, this determination occurring in a spatially resolved manner for the investigation area and the surrounding area; averaging of the determined values of the attenuation parameter in order to obtain an averaged value of the attenuation parameter, this averaging occurring in a spatially resolved manner for the investigation area and the surrounding area; and execution of the attenuation correction of the PET data based on the averaged value for the attenuation parameter.

### BRIEF DESCRIPTION OF THE DRAWINGS

The invention is described in greater detail below on the basis of example embodiments with reference to the drawings. In the diagrams the same reference characters designate identical or similar elements, wherein

FIG. 1 is a schematic view of a combined MRT-PET system;

FIG. 2 illustrates an investigation area of an investigation subject;

FIG. 3 illustrates a Cartesian scanning scheme of the spatial frequency area;

FIG. 4 illustrates a gating technique with reference to a respiration cycle of the investigation subject;

FIG. 5 illustrates a segmentation of MRT data;

FIG. 6 is a flow chart of an embodiment of the inventive method for movement-averaged attenuation correction;

FIG. 7 schematically illustrates the capturing of MRT data with reference to different phases of the respiration cycle of the investigation subject;

FIG. 8 illustrates an averaging of MRT data using already known techniques;

FIG. 9 illustrates an attenuation correction using already known techniques;

FIG. 10 illustrates an averaging of MRT data using inventive techniques; and

FIG. 11 illustrates an attenuation correction using inventive techniques.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

According to one aspect of at least one embodiment, the invention relates to a method for movement-averaged attenuation correction of positron emission tomography (PET) data of an investigation subject, based on magnetic resonance tomography (MRT) data. The method comprises the capture of multiple MRT data respectively for the investigation area and for an area surrounding the investigation area, in each case in different phases of a cycle of an anatomical disposition of the investigation subject. The method furthermore comprises, for each of the plurality of MRT data respectively, the determination of a value of an attenuation parameter from the respective MRT data by way of segmentation, this determination taking place in a spatially resolved manner for the investigation area and the surrounding area. The method furthermore comprises the averaging of the determined values of the attenuation parameter in order to obtain an averaged value of the attenuation parameter, the averaging taking place in a spatially resolved manner for the investigation area and the surrounding area. The method furthermore comprises the execution of attenuation correction of the PET data, based on the averaged value for the attenuation parameter.

For example, the cycle of an anatomical disposition may be the respiration cycle of the investigation subject; in this case e.g. a position of the various anatomical elements of the investigation subject, in particular a position of the diaphragm or lungs, may vary cyclically. It is generally possible, however, for each cyclical movement to be taken into account as an anatomical disposition cycle, e.g. swallowing reflex, etc.

For example, the method may furthermore comprise the capture of PET data for the investigation area of the investigation subject. For example, the method may in particular be executed with a combined PET-MRT system; in such a case it may be possible to capture the PET data and the MRT data without repositioning the investigation subject. It is however also possible to execute the method by way of an MRT system without PET functionality. It may then be possible to execute the capture of the PET data in a separate stage (e.g. by repositioning of the investigation subject between the MRT system and the PET system). In particular, in such a case there may be a significant time delay between the capturing of PET data and the capturing of MRT data.

For example, the MRT data may be captured by way of a Dixon-type MRT measurement sequence or by way of chemical-shift-imaging MRT measurement sequences, which allow various spin species to be inferred in the investigation subject from the MRT data. This is because different MRT parameters such as resonance frequency or relaxation time may have a dependency on the chemical environment of the nuclear spin and may therefore differ, e.g. in particular for fat, water, etc. In this way it is possible to differentiate, for example, between fat tissue, lungs, air and water, and to carry out a segmentation and determination of the value of the attenuation parameter based on this differentiation. Alternatively, a different technique for determining the attenuation parameter may also be executed, for example the techniques by J. Nuyts et al. or A. V. Bronnikov mentioned at the start, the entire contents of each of which are incorporated herein by reference.

Attenuation correction and chemical-shift-imaging techniques are known generally to the person skilled in the art, so that no further details on carrying out the attenuation correction need to be specified here.

At least one embodiment of the invention is based on the finding that the PET data can be captured over a longer period and can be averaged over this longer period. The PET data may therefore be blurred and/or averaged by periodic movement, such as e.g. the respiration cycle of the investigation subject. In contrast, a spatial resolution of the MRT data may be determined mainly by the point in time at which the MRT data is captured for small wave vectors (k-vector), i.e. in a center of the spatial frequency area (k-space). This may be because the MRT data is captured initially in the k-space, and is only then transformed by way of a Fourier transformation into the position space. In other words the spatial information of the MRT data may typically be a static snapshot of a specific point in time, if data samples are captured in the center of the k-space. If MRT data of a moving object (for example the respiring investigation subject) is captured over a relatively long period, e.g. by means of a conventional Cartesian scanning scheme of the k-space, this need not necessarily result in blurred and averaged MRT data (as frequently in the case of PET data), but rather in non-quantifiable MRT data with so-called ghosting artifacts, etc.

Therefore it is possible, according to at least one embodiment of the invention, for the multiple MRT data to be captured respectively in different phases of the respiration cycle of the investigation subject. The different MRT data may then have only minor movement artifacts or none at all. In other words, according to the invention provision may be made for a multiphase series of MRT data to be captured initially (i.e. in different phases of the respiration cycle of the investigation subject respectively), for this data to be converted into attenuation maps (μ-maps) using an appropriate segmentation technique, and only then for these attenuation maps to be averaged, in order to obtain an averaged attenuation map. This attenuation map may correspond to the spatially resolved information about the value of the attenuation parameter.

This may have advantages, particularly in contrast to techniques which average the MRT data first and then determine the attenuation map and/or the averaged value of the attenuation parameter on the basis of the averaged MRT data. A particularly accurate movement-averaged attenuation correction may for example then be achieved if the determined values of the attenuation parameter with regard to the respiration cycle of the investigation subject are based on a comparable anatomical situation, like the captured PET data. In such a case, undercorrection or overcorrection of the PET data in peripheral areas of objects, which are particularly heavily influenced e.g. by the respiration cycle or the swallowing reflex, can be reduced or prevented. In particular, a preceding determination of the attenuation parameter, e.g. individually for each of the plurality of MRT data and subsequent averaging of the determined values, may deliver a different result compared to the reverse situation, in which the MRT data is averaged first and the attenuation parameter is then determined based on averaged MRT data. The result in the inventive first case may in particular deliver results which coordinate more closely with the anatomical situation that forms the basis for the PET data. Continuous movement-averaged data records of the attenuation parameter and of the PET data may therefore be present.

The multiple MRT data may be captured respectively with a Dixon-type MRT-measurement sequence, in which the phasing of the magnetization in fat and water is used at an echo time, in order to differentiate at least between fat and water proportions in the investigation area and the surrounding area. It is possible in addition to differentiate e.g. between lungs and air. Dixon-type MRT measurement sequences may comprise e.g. a plurality of echoes, i.e. may involve a so-called multi-echo MRT-measurement sequence. Such multi-echo MRT measurement sequences may comprise e.g. the capturing of 2 or 3 or more echoes. Alternative techniques are known as chemical-shift imaging, since they are based on movement of the resonances of the nuclear spin depending on the chemical environment. It is also possible alternatively to use corresponding MRT measurement sequences which permit the separation of fat and water.

The method may for example furthermore comprise: determination of further data which describes the anatomical disposition cycle of the investigation subject, the capturing of the multiple MRT data being achieved respectively for different phases of the anatomical disposition cycle via a gating technique that takes the further data into account.

Gating techniques may comprise a triggering or, in general terms, a synchronization of the capturing of MRT data to the further data; so that it may be ensured that the multiple MRT data is captured respectively in the phases of the anatomical disposition cycle that are well defined yet different from one another. Gating techniques are in principle known to the person skilled in the art, so that no further details need to be explained here.

For example, the further data may describe an amplitude of the anatomical disposition cycle, and the different phases of the anatomical disposition cycle, in which the multiple MRT data is captured, may be determined respectively via amplitudes of the anatomical disposition cycle that are equal within tolerance intervals.

In other words, the different phases of the anatomical disposition cycle may be assigned for example via the amplitude of the anatomical disposition cycle. It is therefore also possible e.g. with the phasing of the anatomical disposition cycle shifted by 180°, but having the same amplitude (e.g. ascending and descending edges, for example during inhalation and exhalation), for the same MRT data of the multiple MRT data to be captured. This may be because an anatomical disposition of the investigation subject may be identical in both cases. The multiple MRT data may therefore in particular be captured at the same amplitudes of respiration of the investigation subject.

The gating technique may be selected from the following group: prospective gating technique and retrospective gating technique. The determination of the further data may be executed by means of techniques selected from the following group: measurement of the further data by means of a respiration pillow; and measurement of the further data by means of navigator-MRT data; and determination of the further data from the MRT data using self-gating techniques.

For example the navigator-MRT data with low spatial resolution, i.e. with low measurement duration but with a high repeat rate, can map a diaphragm of the investigation subject; the further data can be determined from this. The respiration amplitude can then be determined for example from the position of the diaphragm. It is possible for the navigator-MRT data to be captured in between multiple the MRT data, e.g. in a so-called interleaved technique.

Various further alternative techniques for determining the further data are known to the person skilled in the art. For example, optical systems may be used for mapping any movement of the ribcage of the investigation subject in order to measure the further data. The respiration pillow may be placed in contact with the ribcage of the investigation subject, and conclusions on respiration amplitude may be drawn from the movement of the respiration pillow.

So-called self-gating techniques may allow conclusions on the anatomical disposition cycle to be drawn from the multiple MRT data. This may be possible if the anatomical situation in the investigation area allows conclusions on the anatomical disposition cycle. The prospective gating technique allows data to be allocated to the different phases of the cycle in real time during the capturing of the multiple MRT data, and therefore the capturing of the multiple MRT data to be controlled to this effect. In contrast, a retrospective gating technique may carry out an allocation to the different phases after the capture of the multiple MRT data; certain parts of the multiple MRT data may then e.g. be discarded, since their allocation to the various different phases of the anatomical disposition cycle does not fit into the gating scheme. The prospective gating technique may therefore allow discrimination between the different phases of the respiration cycle of the investigation subject, even during the capturing of the MRT data. In other words the respective phase of the respiration cycle may be determined even during the capturing of the multiple MRT data, and the capturing of the multiple MRT data may then be carried out selectively within so-called gating windows. In contrast to this, in the retrospective gating technique such parts of the MRT data that were captured during incorrect phases of the respiration cycle of the investigation subject may be retrospectively discarded.

In each case it may be desirable to capture the multiple MRT data with scanning schemes of the k-space, which allow a combination with the most diverse gating techniques. For example it may be possible to capture the multiple MRT data by means of a Cartesian scanning scheme of the k-space. It may then be possible to capture each of the plurality of MRT data block by block or in segments, in order thus to capture e.g. the different quadrants of the k-space sequentially in different cycles of the respiration cycle of the investigation subject, for example respectively at intervals of complete periods of the anatomical disposition cycle. The time needed to capture a segment of the respective MRT data may then be comparatively lower than the time for capturing the complete k-space, so that it may be possible to ensure, e.g. even during rapid respiration, that the various data of the multiple MRT data is captured in respectively different and well-defined phases of the respiration cycle of the investigation subject. Corresponding techniques are known to the person skilled in the art, so that no further details need to be explained here.

Reference has been made above especially to techniques which permit the multiple MRT data to be captured respectively in different phases of the anatomical disposition cycle. The averaging of the determined values of the attenuation parameter will be discussed below in particular.

For example, the averaging can take into account averaging weightings for the various determined values of the attenuation parameter, the averaging weightings being determined based on the anatomical disposition cycle.

The averaging weightings may for example take into account properties of the anatomical disposition cycle. For example, the averaging may thus take into account a frequency of occurrence of the respective different phases of the anatomical disposition cycle. It is for example possible in particular for deep and shallow respiratory phases, i.e. large and small amplitudes of the respiration cycle, to be taken into account via the averaging weightings.

It is possible, for example, that the averaging weighting that corresponds to the attenuation parameter of determined MRT data of the multiple MRT data, corresponds to a fraction of the duration for the capturing of determined MRT data in the duration for the capturing of the entire plurality of MRT data. In other words the attenuation parameters can be weighted according to the respiration curve during averaging. More (or less) frequently occurring amplitudes of the anatomical disposition cycle may be weighted more (or less) heavily.

According to a further aspect, at least one embodiment of the invention relates to an MRT system for movement-averaged attenuation correction of PET data of an investigation area of an investigation subject, based on MRT data, the MRT system comprising an MRT imaging unit and a processor unit. The MRT imaging unit is configured to capture multiple MRT data respectively for the investigation area and an area surrounding the investigation area and respectively in different phases of a cycle of the anatomical disposition of the investigation subject. The processor unit is configured to carry out the following steps: respectively for each of the plurality of MRT data: determination of a value of an attenuation parameter from the respective MRT data by means of segmentation, this determination occurring in a spatially resolved manner for the investigation area and the surrounding area; averaging of the determined values of the attenuation parameter in order to obtain an averaged value of the attenuation parameter, this averaging occurring in a spatially resolved manner for the investigation area and the surrounding area; and execution of the attenuation correction of the PET data based on the averaged value for the attenuation parameter.

Effects may be obtained for such an MRT system that are comparable to the effects that may be obtained for a method for movement-averaged attenuation correction according to a further aspect of at least one embodiment of the present invention.

The features of the previously described embodiments and aspects of the invention may of course be combined with one another. In particular the features may be used not only in the described combinations, but also in other combinations or taken in isolation, without departing from the scope of the invention.

Example embodiments of the invention, which allow a particularly precise and at the same time particularly simple attenuation correction of PET data based on MRT data, are described below on the basis of the figures. From multiple MRT data captured in different phases of a respiration cycle of an investigation subject, attenuation parameters can be determined respectively by means of segmentation and these can then be averaged. This may allow a more accurate attenuation correction. The following description of embodiments with reference to the figures should not be interpreted in a limited way. The figures are purely illustrative.

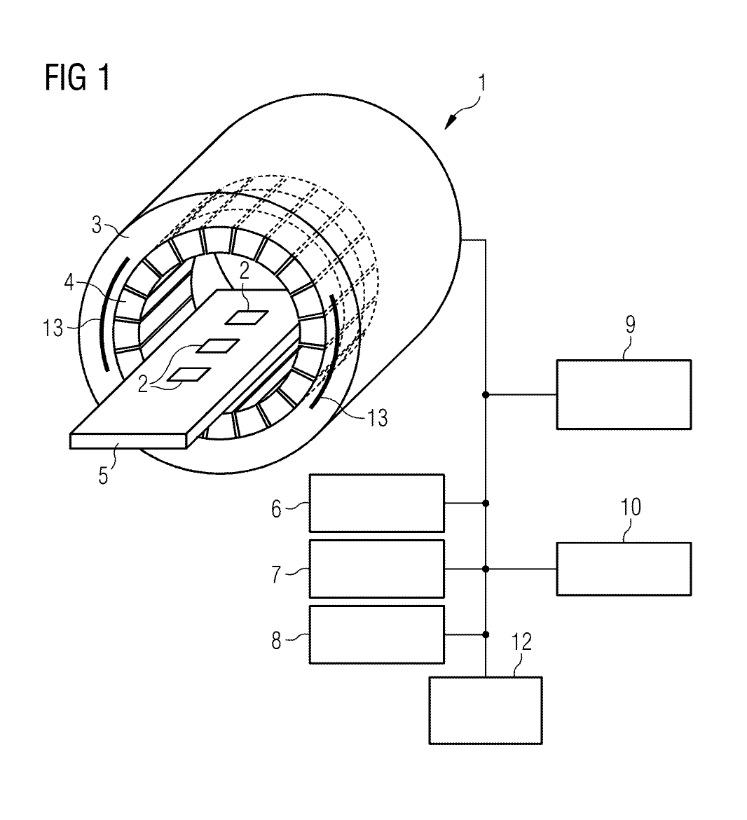

FIG. 1 is a schematic illustration of a combined PET-MRT system 1. An investigation subject may be placed on a table 5 and positioned inside a magnet 3. The magnet 3 may create a static (DC) magnetic field up to a level of a few Tesla, in order to align nuclear spin in the investigation subject. The magnet 3 may comprise superconductive coils in liquid helium.

A PET imaging unit 8 and a PET detector 4 are provided; they are configured to capture PET data. The PET detector 4 measures coincidental events of PET photons and the PET imaging unit 8 provides the PET data based on these measurements. Details of the operation of the PET components 4, 8 are known to the person skilled in the art, so that there is no need to discuss further details in this connection.

An MRT-imaging unit 7 and an MRT detector 2 are configured to execute MRT measurement sequences and provide MRT data. The MRT detector 2 comprises high-frequency (HF) coils for exciting and for detecting a magnetization dynamic of the nuclear spin. Gradient coils 13 for position encoding of the MRT data by way of gradient fields are also provided. The operation of the MRT components 2, 7, 13 as an MRT system is known to the person skilled in the art, so that there is no need to discuss further details in this context.

In particular, the MRT imaging unit 7 is configured such that such MRT data is provided that is indicative of a linear attenuation parameter of the PET photons. This may occur through corresponding MRT measurement sequences, e.g. by means of a Dixon-type measurement sequence or related techniques. The MRT data may then be used by a processor unit 10, in order to carry out an attenuation correction of the PET data.

In order to carry out the attenuation correction with a high level of accuracy, i.e. with very few errors, the processor unit 10 is additionally configured to provide further data that is indicative for the respiration cycle of the investigation subject. In other words the further data may describe the respiration cycle of the investigation subject, e.g. by describing the amplitude of the respiration cycle of the investigation subject. It is optionally possible for a respiration unit 6 to be provided, which is configured for measuring the further data that describes the respiration cycle of the investigation subject.

In addition the PET-MRT system 1 comprises a user interface 12, which permits input and output to a user. For example, the various operating parameters of the PET-MRT system 1 may be controlled by way of the user interface 12. The user interface 12 may comprise, in different embodiments, a keyboard, a screen, a mouse or other input devices.

The PET-MRT system 1 further comprises a gating unit 9, which is configured to allow the capture of MRT data by way of the MRT detector 2 and the MRT imaging unit 7 taking into account a gating technique. In particular, the gating unit 9 is linked to the processor unit 10 so that, based on the further data that is indicative of the respiration cycle of the investigation subject, the gating unit 9 may control the capture of multiple MRT data respectively in different phases of the respiration cycle of the investigation subject. Particularly in the context of prospective gating techniques, the multiple MRT data may be captured in each case based on the further data, triggered at such times to correspond to different phases of the respiration cycle.

The gating unit 9 may for example be configured to execute a prospective gating technique or a retrospective gating technique. In a prospective gating technique, the multiple MRT data is captured by the MRT imaging unit 7 already synchronized to the respiration cycle; in the retrospective gating technique, certain parts of the captured MRT data which are outside the corresponding gating windows, i.e. were captured in incorrect phases of the respiration cycle of the investigation subject, are retrospectively discarded. Corresponding techniques are generally known to the person skilled in the art, so that it is not necessary to discuss further details of the gating unit 9 in this connection.

The respiration unit 6 may optionally be provided. For example, the respiration unit 6 may be a respiration pillow which is placed in contact with the ribcage of the investigation subject and measures any movement during inhalation and exhalation. On the basis of this measurement, it is possible for example for the amplitude of the respiration cycle to be determined in a time-resolved manner. Alternatively, the respiration unit 6 may be part of the MRT imaging unit 7 and the further data may be determined by means of special navigator-MRT data. The navigator-MRT data may for example map the diaphragm of the investigation subject and may therefore be indicative of the amplitude of the respiration cycle of the investigation subject. The person skilled in the art will be aware of further techniques for determining the further data, so that for example an optical mapping of the respiration of the investigation subject or a self-gated technique in which a conclusion on the respiration cycle of the investigation subject may be drawn e.g. from the MRT data itself.

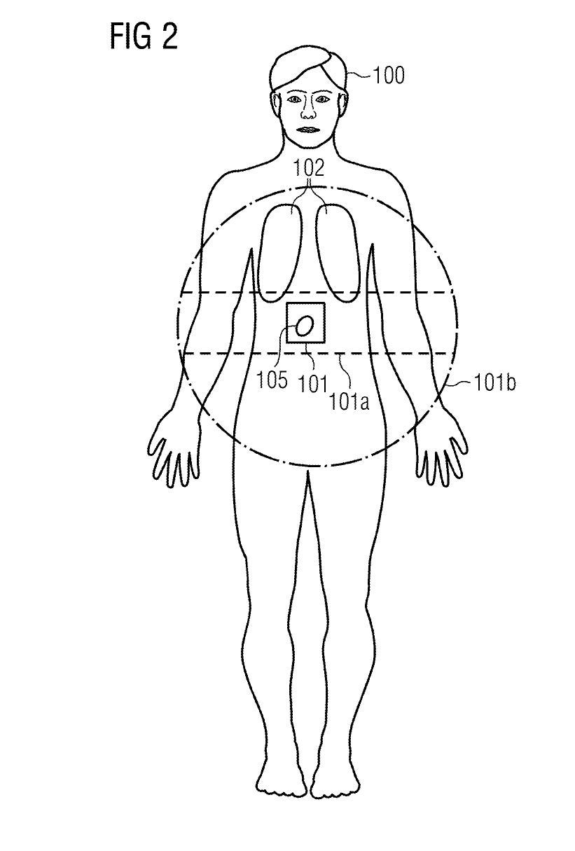

With reference to FIG. 2: FIG. 2 illustrates the investigation subject 100, as well as an organ 105 and the lungs 102 of the investigation subject 100. The aim may be, for example, to generate images of the organ 105 by way of PET. The organ 105 is therefore located in the investigation area 101. Also illustrated (indicated in FIG. 2 with a broken line) is an area 101a surrounding the investigation area 101. A field of view 101b of the combined PET-MRT system 1 (shown in FIG. 2 with a dotted and broken line) is shown in addition. By capturing the MRT data both in the investigation area 101 and in the surrounding area 101a, an attenuation correction can be guaranteed for the entire travel path of PET photons within the investigation subject 100.

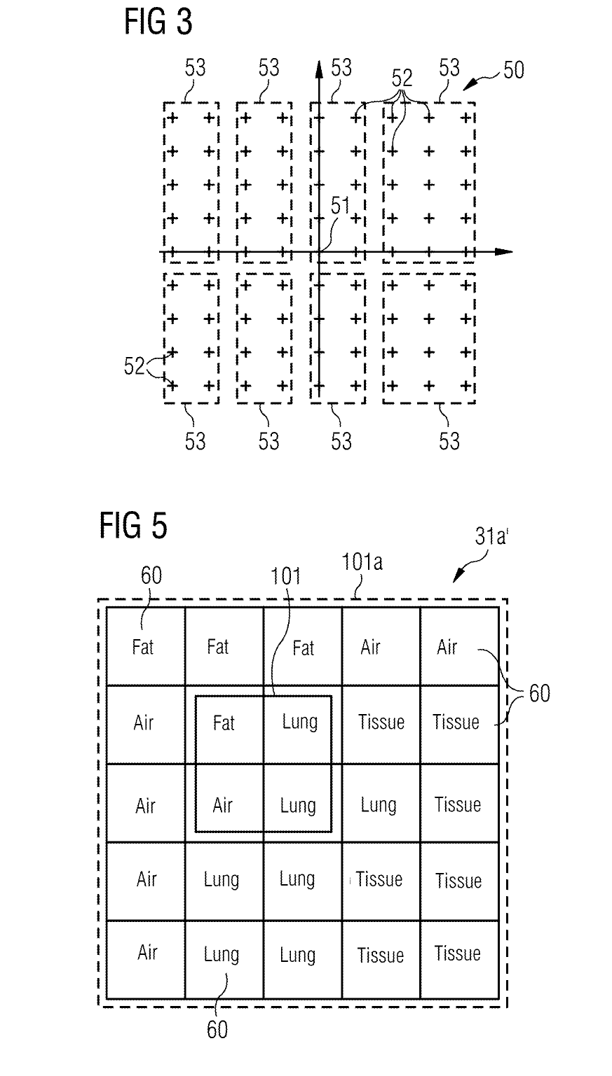

FIG. 3 shows the spatial frequency area 50 (k-space) with scanning points 52 (k-space samples), which are assigned in a Cartesian scheme. A center 51 of the k-space 50 is shown. A spatial allocation of MRT data 50 is essentially determined by those scanning points 52 which are assigned near the k-space center 51 (low-frequency). In contrast to the PET data which is already captured directly in the position space, special movement artifacts, e.g. ghosting artifacts, are therefore characteristic of this capturing of the MRT data in the k-space 50.

The MRT imaging unit 7 is configured to scan the k-space 50 in segments 53, which respectively comprise a fraction of all scanning points 52. This means that the time per captured segment may be lower than the time for capturing all scanning points 52 of the entire k-space 50. In combination with the gating unit 9, this may allow an entire segment 53 to be controlled and predictably captured within short gating windows. The MRT data may then be respectively completed from a plurality of sequentially captured segments 53. In particular it may thus be ensured that the multiple MRT data is captured respectively in different phases of the respiration cycle. It is noted that the segments 53 in FIG. 3 are purely exemplary and should not be interpreted in a limited way. Other shapes and dimensions of segments 53 are possible according to an embodiment of the invention.

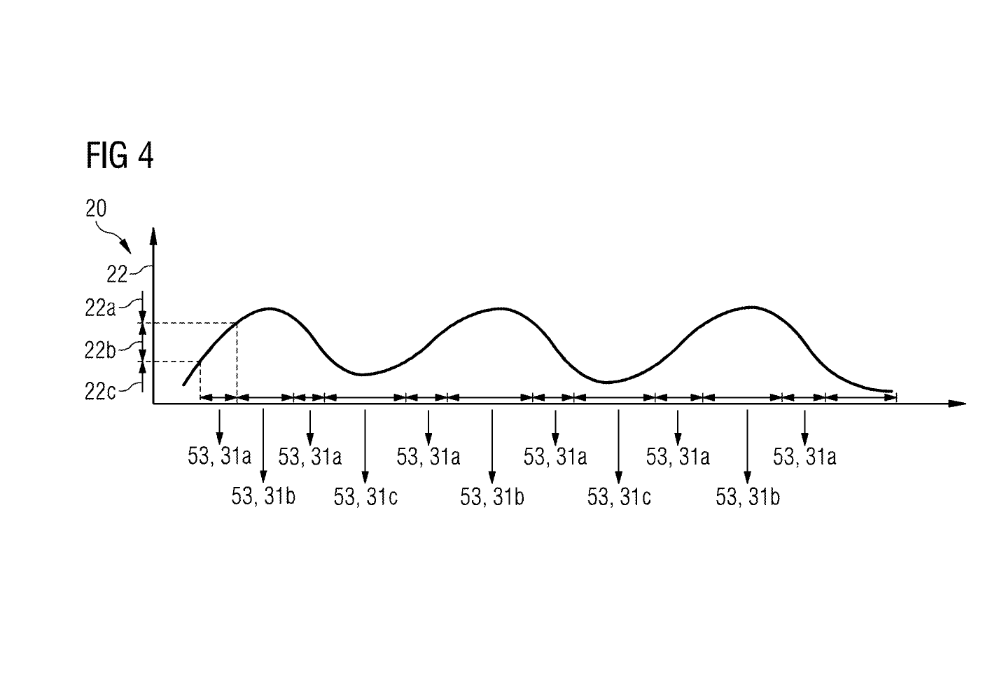

This is illustrated in FIG. 4. FIG. 4 shows the respiration cycle 20 of the investigation subject 100; in particular, the amplitude 22 of the respiration cycle 20 is plotted over time in FIG. 4. By way of the gating technique used by the gating unit 9, the different segments 53 of the k-space 50 are captured, being separated respectively for multiple MRT data 31a-31c. FIG. 4 shows that the different MRT data 31a-31c corresponds respectively to different phases of the respiration cycle 20. For example, the MRT data 31c (31b) is captured at small (large) amplitudes 22 of the respiration cycle 20, while the MRT data 31a is captured at average amplitudes 22 of the respiration cycle 20. In particular, the phases of the respiration cycle 20, in which the MRT data 31a-31c is respectively captured, have the same amplitudes 22 within tolerance intervals 22a-22c (indicated by the broken line in FIG. 4).

The MRT data 31a-31c may then be segmented by the processor unit 10. In other words, each pixel may be assigned a value of the attenuation parameter selected from a defined set. This is illustrated in FIG. 5; fat, air, lungs and tissue are marked for different (known to the person skilled in the art) attenuation parameters 60 for the investigation area 101 and the surrounding area 101a for illustrative purposes in FIG. 5. This may be based e.g. on a 3 or 4-point multi-echo Dixon-type MRT measurement sequence, as is known to the person skilled in the art. Such segmented MRT data 31a′ may be used for attenuation correction. The illustration in FIG. 5 corresponds to an attenuation parameter map.

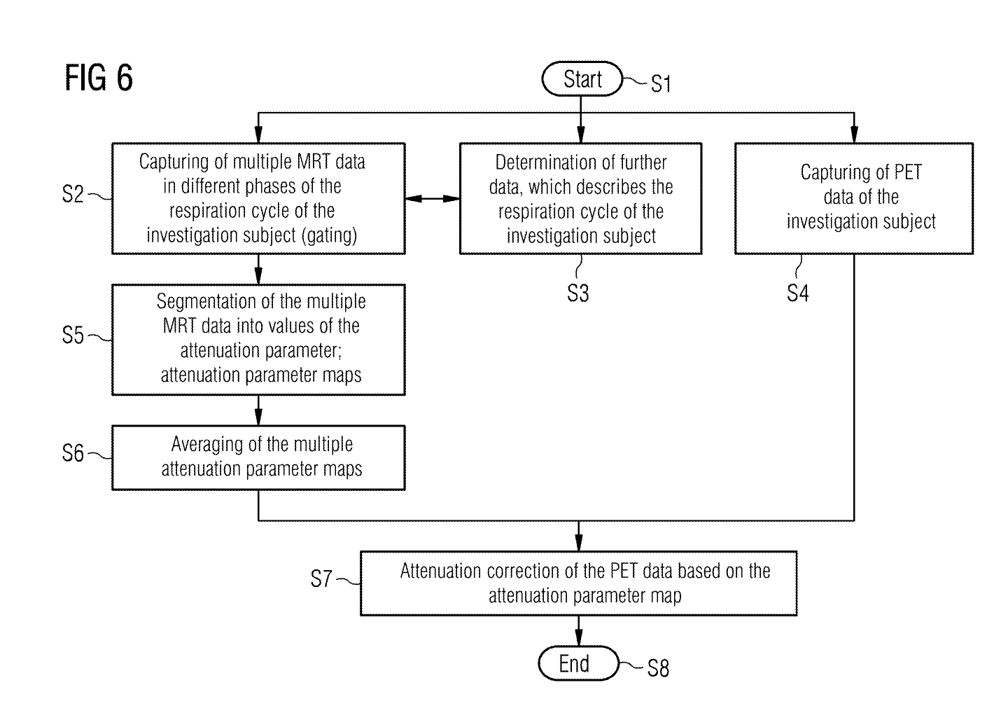

The process steps of an inventive method for attenuation correction are shown in FIG. 6.

The method begins in step S1. In step S2 the multiple MRT data 31a-31c is captured respectively in different phases of the respiration cycle 20 of the investigation subject 100. For example, the capturing may take place in the different phases of the respiration cycle 20 by means of a previously described gating technique using the gating unit 9. This gating technique may comprise e.g. in step S3 the measurement and/or determination of further data, which describes the respiration cycle 20 of the investigation subject 100, for example by way of the respiration unit 6 and the processor unit 10. For example, the further data may describe an amplitude 22 of the respiration cycle 20 of the investigation subject 100. In prospective gating techniques a direct link may therefore exist between steps S2 and S3 (graphically indicated in FIG. 5 by the horizontal double arrow) and the capturing of the multiple MRT data 31a-31c may be triggered respectively based on the further data.

The PET data of the investigation subject 100 is captured in step S4. In cases involving the combined MRT-PET system 1, as discussed previously, the capturing of the multiple MRT data in step S2 and the capturing of the PET data in step S4 may take place at least partially simultaneously. Alternatively, it is also possible for the capturing of PET data to be carried out at a different time, e.g. with a separate PET system.

The segmentation of the multiple MRT data 31a-31c respectively into values of the attenuation parameter subsequently takes place in step S5. This takes place in a spatially resolved manner and may therefore comprise the provision of an attenuation parameter map respectively for each of the plurality of MRT data 31a-31c.

This is followed in step S6 by the averaging of the multiple attenuation parameter values. The averaging of the multiple values of the attenuation parameter 60 may take averaging weightings into account. The averaging weightings may be determined on the basis of the respiration cycle 20. For example, it is possible for the averaging weightings to be proportional to the time spans, indicated in FIG. 4 with horizontal arrows, which are available for capturing the respective MRT data 31a, 31b, 31c. More frequently (rarely) occurring amplitudes 22 are therefore taken into account to a greater (lesser) extent. The absorption correction of the PET data based on the averaged values of the attenuation parameter, as determined in step S6, is carried out in step S7.

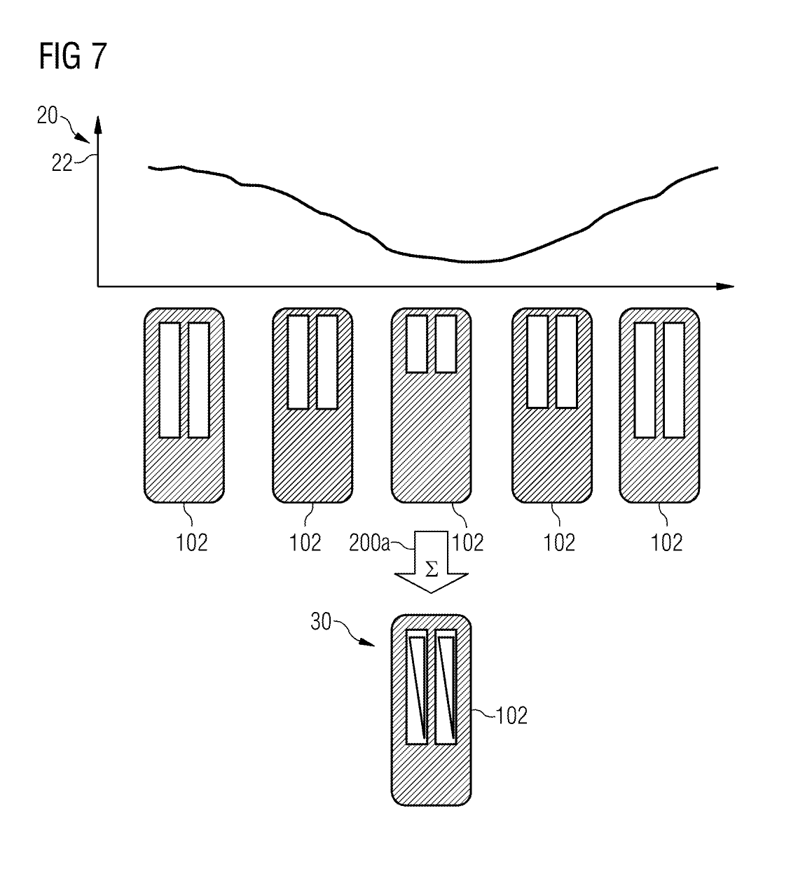

It may be seen from FIG. 6 and the above description that the value of the attenuation parameter is first determined by way of segmentation in step S6 and the attenuation parameters are then averaged. This may allow a particularly accurate attenuation correction in step S7. The way in which such a technique allows particularly accurate attenuation correction is described below with reference to FIGS. 7-11.

A period of a respiration cycle 20 is shown at the top of FIG. 7. FIG. 7 indicates in particular the amplitude 22 of the respiration cycle 20. The state of the lungs 102 of the investigation subject 100 in each of the different phases of the respiration cycle 20 is illustrated schematically in the center of FIG. 7.

PET events mapping the lungs 102 are measured respectively for five different phases of the respiration cycle 20, i.e. the PET data 30 is composed from an averaging of these five PET events. This is illustrated schematically in FIG. 7 by the vertical arrow. The PET data 30 contains in particular a gradual contrast gradient. The greatest contrast is obtained in those areas of the investigation subject 100 in which the lungs 102 are disposed even during exhalation (at the top of FIG. 7 respectively). The lowest contrast is obtained where the lungs 102 are extended only during full inhalation, i.e. marked by the maximum volume of the lungs 102 (at the bottom of FIG. 7 respectively). This contrast gradient in the PET data 30 is illustrated schematically at the bottom of FIG. 7 by the downward pointing triangles in the lungs 102.

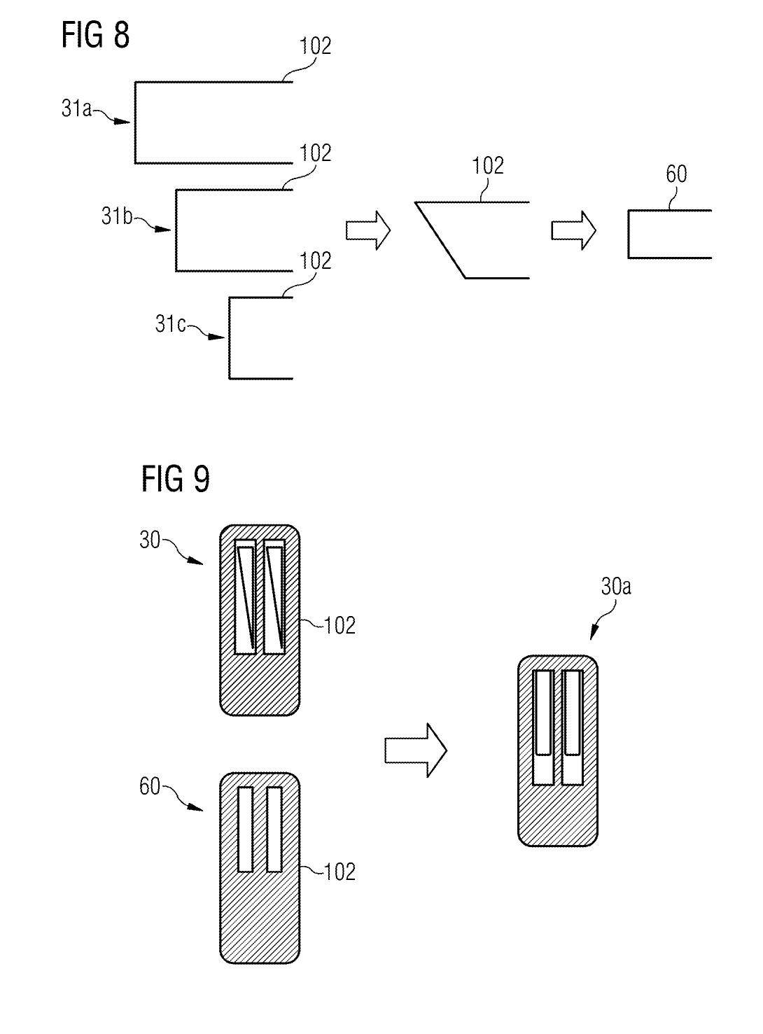

Multiple MRT data 31a, 31b, 31c is illustrated schematically on the left of FIG. 8, such data being captured respectively at different phases of the respiration cycle 20. The MRT data 31a, 31b, 31c therefore shows in each case a different expansion of the lungs 102 (illustrated schematically in FIG. 8 by the boxes). The averaging of the MRT data 31a-31c then takes place in FIG. 8 (first horizontal arrow) and the value of the attenuation parameter 60 for the averaged MRT data is then determined (second horizontal arrow). Such a technique, which comprises first the averaging and then the segmentation, is not part of the invention. The segmentation may include e.g. a threshold comparison, which assigns two discrete values of the attenuation parameter 60 to the continuous progress of the averaged MRT data (illustrated in the center of FIG. 8).

A technique described above (which is not an object of the invention) produces the attenuation correction situation shown in FIG. 9: the PET data 30 is attenuation-corrected by means of the values of the attenuation parameter 60 which were determined using the technique shown in FIG. 8 (illustrated in FIG. 9 by way of the horizontal arrow). This results in attenuation-corrected PET data 30a. As can be seen from FIG. 9, there is an overcorrection or undercorrection of the PET data 30 particularly in peripheral areas of the lungs 102 of the investigation subject 100. This is particularly because the lungs 102 are not identically mapped in the PET data 30 and the values of the absorption parameter 60. This is a result of the technique previously described with reference to FIG. 8, in which an averaging takes place first, followed by a segmentation.

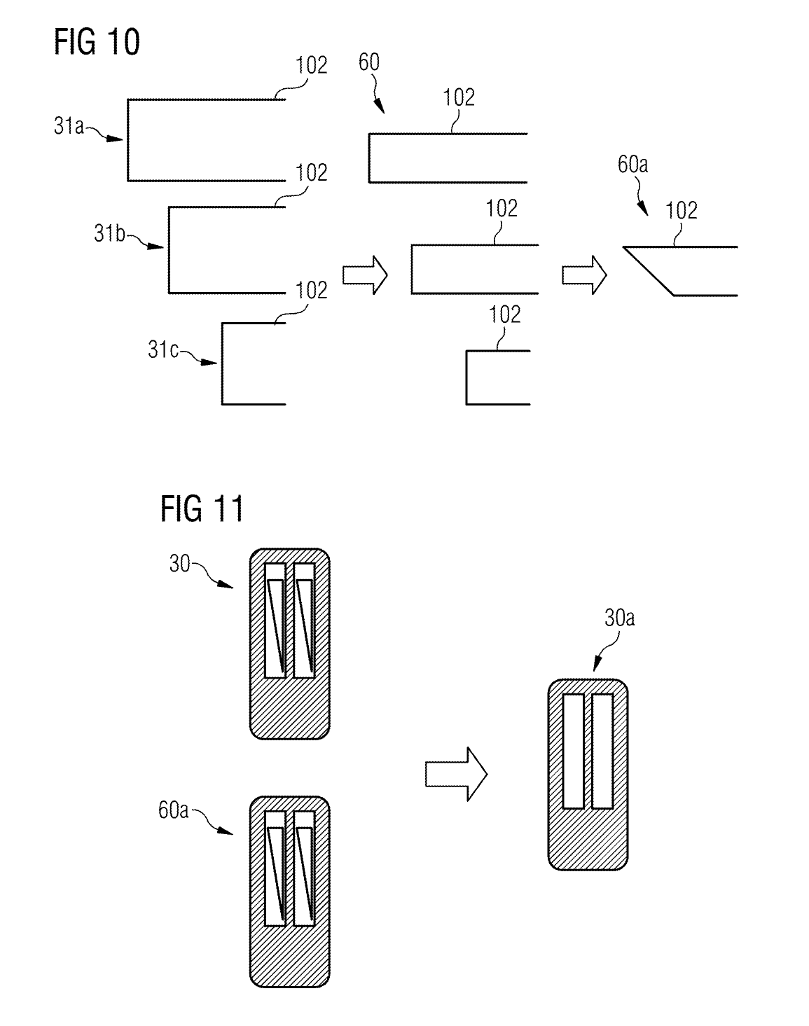

An inventive method for movement-averaged attenuation correction is illustrated next, with reference to FIG. 10. The starting point is again the multiple MRT data 31a-31c, which maps the lungs 102 of the investigation subject 100. In the embodiment of the invention as shown in FIG. 9, firstly the values of the attenuation parameter 60 are determined, or the attenuation parameter map is determined (shown in FIG. 10 by the first horizontal arrow). This corresponds to the segmentation of the MRT data 31a, 31b, 31c. The multiple values of the attenuation parameter 60 are then averaged. Since the averaging is not a discrete operation, a gradual progression of the determined values of the attenuation parameter 60 may result.

This situation is graphically indicated in FIG. 11 by the decreasing triangles in the lungs 102. In FIG. 11 the lungs 102 are shown to be comparable in the PET data 30 and in the values of the absorption parameter 60—this is a result of the preceding segmentation and subsequent averaging (see FIG. 10); comparably accurate attenuation-corrected PET data 30a may therefore be obtained.

Even though the invention has been illustrated and described in greater detail by the preferred embodiments, the invention is not restricted by the disclosed examples and other variations may be derived therefrom by the person skilled in the art, without departing from the scope of protection of the invention.

For example, particular reference has been made above to a combined MRT-PET system. However, this should be interpreted as meaning that corresponding techniques may also be used for an MRT system without PET functionality. It may then be possible, for example, for the PET data to be captured in a separate process step in a separate PET system. This may involve the repositioning of the investigation subject.

In addition, particular reference has been made in the diagrams to a respiration cycle of the investigation subject. However, this should be interpreted as meaning that corresponding inventive techniques may be applied generally to each cycle of an anatomical disposition, e.g. even swallowing, etc.

Particular reference has furthermore been made to segmentation processes, which allow allocation of values of the attenuation parameter for PET data to MRT data. In general, however, inventive techniques may be applied to all techniques for image segmentation. This relates e.g. in particular to image segmentation in the context of object recognition.

What is claimed is:

1. A method for the movement-averaged attenuation correction of positron emission tomography (PET) data of an investigation area of an investigation subject, based on magnetic resonance tomography (MRT) data, the method comprising:
capturing of multiple MRT data respectively for the investigation area and an area surrounding the investigation area and respectively in different phases of a cycle of an anatomical disposition of the investigation subject;
determining, respectively for each of the plurality of MRT data, a value of an attenuation parameter from the respective MRT data via segmentation, the determination taking place in a spatially resolved manner for the investigation area and the surrounding area;
averaging the determined values of the attenuation parameter to obtain an averaged value of the attenuation parameter, the averaging taking place in a spatially resolved manner for the investigation area and the surrounding area; and
executing the attenuation correction of the PET data based on the averaged value of the attenuation parameter.

2. The method of claim 1, further comprising:
determining further data, which describes the cycle of the anatomical disposition of the investigation subject, wherein the capturing of the multiple MRT data is achieved respectively for the different phases of the cycle of the anatomical disposition via a gating technique which takes the further data into account.

3. The method of claim 2, wherein the further data describes an amplitude of the cycle of the anatomical disposition and wherein the different phases of the cycle of the anatomical disposition, in which phases the multiple MRT data is captured, are determined respectively via identical amplitudes of the cycle of the anatomical disposition within tolerance intervals.

4. The method of claim 2, wherein the gating technique is selected from the following group:
prospective gating technique, and
retrospective gating technique; and
wherein the determination of the further data is carried out by way of techniques selected from the following group:
measurement of the further data by way of a respiration pillow,
measurement of the further data by way of navigator-MRT data, and
determination of the further data from the MRT data using self-gating techniques.

5. The method of claim 1, wherein the averaging of averaging weightings provides for the different determined values of the attenuation parameter, wherein the averaging weightings are determined based on the cycle of the anatomical disposition.

6. The method of claim 3, wherein the averaging weighting that corresponds to the attenuation parameter of defined MRT data of the multiple MRT data, corresponds to a fraction of the duration for the capturing of the determined MRT data in the duration for the capturing of the entire plurality of MRT data.

7. The method of claim 1, wherein the capturing of the multiple MRT data takes place respectively with a Cartesian scanning of the spatial frequency area.

8. The method of claim 1, wherein the capturing of the multiple MRT data takes place respectively with a Dixon-type MRT measurement sequence, in which the phasing of the magnetization in fat and water is used at an echo time, in order to differentiate at least between fat and water proportions in the investigation area and in the surrounding area.

9. An MRT system for the movement-averaged attenuation correction of positron emission tomography (PET) data of an investigation area of an investigation subject, based on magnetic resonance tomography (MRT) data, the MRT system comprising:
an MRT imaging unit, configured to capture multiple MRT data respectively for the investigation area and an area surrounding the investigation area, respectively in different phases of a cycle of an anatomical disposition of the investigation subject; and
a processor unit, configured to carry out at least the following steps:
determining, respectively for each of the plurality of MRT data, a value of an attenuation parameter from the respective MRT data via segmentation, wherein the determination takes place in a spatially resolved manner for the investigation area and the surrounding area,
averaging the determined values of the attenuation parameter to obtain an averaged value of the attenuation parameter, wherein the averaging takes place in a spatially resolved manner for the investigation area and the surrounding area, and
executing the attenuation correction of the PET data based on the averaged value of the attenuation parameter.

10. The method of claim 3, wherein the gating technique is selected from the following group:
prospective gating technique, and
retrospective gating technique; and
wherein the determination of the further data is carried out by way of techniques selected from the following group:
measurement of the further data by way of a respiration pillow,
measurement of the further data by way of navigator-MRT data, and
determination of the further data from the MRT data using self-gating techniques.

11. The MRT system of claim 9, wherein the processor is further configured to determine further data, which describes the cycle of the anatomical disposition of the investigation subject, wherein the capturing of the multiple MRT data is achieved respectively for the different phases of the cycle of the anatomical disposition via a gating technique which takes the further data into account.

12. The MRT system of claim 10, wherein the further data describes an amplitude of the cycle of the anatomical disposition and wherein the different phases of the cycle of the anatomical disposition, in which phases the multiple MRT data is captured, are determined respectively via identical amplitudes of the cycle of the anatomical disposition within tolerance intervals.

13. The MRT system of claim 10, wherein the gating technique is selected from the following group:
prospective gating technique, and
retrospective gating technique; and
wherein the determination of the further data is carried out by way of techniques selected from the following group:
measurement of the further data by way of a respiration pillow,
measurement of the further data by way of navigator-MRT data, and
determination of the further data from the MRT data using self-gating techniques.

14. The MRT system of claim 9, wherein the averaging of averaging weightings provides for the different determined values of the attenuation parameter, wherein the averaging weightings are determined based on the cycle of the anatomical disposition.
